(19) United States
(12) Patent Application Publication
(10) Pub. No.: US 2022/0184390 A1
(43) Pub. Date: Jun. 16, 2022

(54) CONDUCTIVE EYE MASK SKIN TREATMENT DEVICE

(71) Applicants: Pooja Johari, Carlsbad, CA (US); Tejas Shah, Carlsbad, CA (US)

(72) Inventors: Pooja Johari, Carlsbad, CA (US); Tejas Shah, Carlsbad, CA (US)

(21) Appl. No.: 17/552,244

(22) Filed: Dec. 15, 2021

Related U.S. Application Data
(60) Provisional application No. 63/126,373, filed on Dec. 16, 2020.

(51) Int. Cl. A61N 1/32; A61N 1/04; A61N 1/36

(52) U.S. Cl. CPC A61N 1/328; A61N 1/36014; A61N 1/0452; A61N 1/0496

(57) ABSTRACT

An apparatus and methods are provided for a conductive eye mask that may be used in conjunction with a handheld electrostimulation skin treatment device for skin toning and firming. The conductive eye mask comprises an eye mask for being applied around the eyes of a practitioner. Electrode pads are attached to an underside of the eye mask such that each of the electrode pads at least partially surrounds each of the practitioner's eyes. The electrode pads include an adhesive for attaching to the skin of the practitioner. An electric connector is coupled with each of the electrode pads. The electric connectors are configured to be plugged into accessory connectors comprising the electrostimulation skin treatment device for sending a microcurrent to the electrode pads to tone and firm the skin and muscles surrounding the practitioner's eyes.

### PRIORITY

This application claims the benefit of and priority to U.S. Provisional Application, entitled “Conductive Eye Mask Skin Treatment Device,” filed on Dec. 16, 2020 and having application Ser. No. 63/126,373, the entirety of said application being incorporated herein by reference.

### FIELD

Embodiments of the present disclosure generally relate to the field of electrostimulation devices. More specifically, embodiments of the disclosure relate to an apparatus and methods for a conductive eye mask that may be used in conjunction with a handheld electrostimulation skin treatment device for skin toning and firming.

### BACKGROUND

Electrostimulation is a well-known technology for non-invasive anti-aging treatments that enables users to age gracefully. Typically, electrostimulation devices tend to include microcurrent devices microcurrent devices that apply currents on the order of 10⁻⁶ amperes to the skin and underlying muscle of a user and millicurrent devices that apply larger currents, on the order of 10⁻³ amperes to the skin and underlying muscles of a user. Millicurrent devices generally apply an electric current to the skin and underlying muscle of a subject to cause involuntary rhythmic contractions of the muscles that improves muscle tone. Microcurrent devices are configured to apply much smaller currents, and thus microcurrent treatments do not cause muscle contractions and tend to be barely detectable by the subject.

Microcurrent treatments are known to increase the amount of adenosine triphosphate (ATP) within the cells of a muscle. For example, experimental demonstrations have shown that an application of a current on the order of 50 to 500 microamperes to the skin and underlying muscle of a subject can give rise to a 300-500% increase in ATP, as well as an increase in mitochondria and protein synthesis in the muscle, an increase in aminoisobutyric acid uptake, and an increase in protein synthesis and Gluconeogenesis (biosynthesis of new glucose). These dramatic increases in cellular ATP levels have been shown to help muscles retain a re-educated form for longer periods of time, and consequently such techniques are of use in muscle toning treatments.

As will be appreciated, for the benefits of muscle toning treatments to be appreciable it is necessary for the muscles to be manipulated, such as by way of extending or compressing the muscles, while the treatments are taking place. In a salon environment, microcurrent electrostimulation devices typically include a pair of probes that can be used by a technician during a treatment to manipulate the skin and muscle such that the muscles are forced into a desired form for re-education. In a home environment, however, subjects desiring microcurrent treatments generally find themselves without the assistance of another person. As such, there is a need for handheld microcurrent electrostimulation devices and accessories that are intended for personally performing muscle toning treatments in absence of the assistance of another person.

### SUMMARY

An apparatus and methods are provided for a conductive eye mask that may be used in conjunction with a handheld electrostimulation skin treatment device for skin toning and firming. The conductive eye mask comprises an eye mask for being applied around the eyes of a practitioner. Electrode pads are attached to an underside of the eye mask such that each of the electrode pads at least partially surrounds each of the practitioner's eyes. The electrode pads include an adhesive for attaching to the skin of the practitioner. An electric connector is coupled with each of the electrode pads. The electric connectors are configured to be plugged into accessory connectors comprising the electrostimulation skin treatment device for sending a microcurrent to the electrode pads to tone and firm the skin and muscles surrounding the practitioner's eyes.

In an exemplary embodiment, a conductive eye mask for treating the skin and muscles surrounding the eyes comprises: an eye mask for being applied around the eyes; one or more electrode pads coupled with the eye mask; and an electric connector coupled with each of the one or more electrode pads.

In another exemplary embodiment, the eye mask includes round strips configured to encircle the eyes and joined at a nose bridge. In another exemplary embodiment, an eye opening is disposed within each round strip to allow a practitioner receiving treatment to see. In another exemplary embodiment, the one or more electrode pads are attached to an underside of the eye mask such that each of the one or more electrode pads at least partially surrounds an eye opening. In another exemplary embodiment, the one or more electrode pads includes an adhesive for attaching to skin of a practitioner receiving treatment. In another exemplary embodiment, the electric connector is configured to be plugged into an accessory connector comprising an electrostimulation skin treatment device for sending a microcurrent to the one or more electrode pads.

In an exemplary embodiment, a method for a conductive eye mask for treating the skin and muscles surrounding the eyes comprises: forming an eye mask for being applied around the eyes; attaching one or more electrode pads with the eye mask; and coupling an electric connector with each of the one or more electrode pads.

In another exemplary embodiment, forming includes configuring round strips to encircles the eye and joining the round strip at a nose bridge. In another exemplary embodiment, attaching includes affixing the one or more electrode pads to an underside of the eye mask such that each of the one or more electrode pads at least partially surrounds an eye opening. In another exemplary embodiment, coupling includes configuring the electric connector to be plugged into an accessory connector comprising an electrostimulation skin treatment device for sending a microcurrent to the one or more electrode pads.

In an exemplary embodiment, a method for treating skin and muscles surrounding the eyes of a practitioner comprises: applying an eye mask around the eyes; attaching one or more electrode pads of the eye mask to the skin; coupling the one or more electrode pads with an electrostimulation skin treatment device; and applying a microcurrent to the one or more electrode pads.

In another exemplary embodiment, applying includes encircling the eyes with round strips comprising the eye mask such that the practitioner can see through eye openings disposed in the eye mask. In another exemplary embodiment, attaching includes using a suitable adhesive to hold the one or more electrode pads in contact with the skin. In another exemplary embodiment, coupling includes plugging the one or more electrode pads into the electrostimulation skin treatment device. In another exemplary embodiment, plugging includes coupling an electric connector comprising the eye mask into an accessory connector comprising an electrostimulation skin treatment device. In another exemplary embodiment, an electric connector is coupled with each of the one or more electrode pads.

These and other features of the concepts provided herein may be better understood with reference to the drawings, description, and appended claims.

### BRIEF DESCRIPTION OF THE DRAWINGS

The drawings refer to embodiments of the present disclosure in which:

FIG. 1 illustrates an exemplary-use environment wherein a handheld electrostimulation skin treatment device is coupled with a conductive eye mask for skin treatment, in accordance with the present disclosure;

FIG. 2 illustrates a top view of an exemplary embodiment of a handheld electrostimulation skin treatment device, in accordance with the present disclosure;

FIG. 3 illustrates a perspective view of an exemplary embodiment of an eye mask that may be coupled with the handheld electrostimulation skin treatment device of FIG. 2, according to the present disclosure; and

FIG. 4 illustrates a perspective view of an underside of the eye mask of FIG. 3 in accordance with the present disclosure.

While the present disclosure is subject to various modifications and alternative forms, specific embodiments thereof have been shown by way of example in the drawings and will herein be described in detail. The present disclosure should be understood to not be limited to the particular forms disclosed, but on the contrary, the intention is to cover all modifications, equivalents, and alternatives falling within the spirit and scope of the present disclosure.

### DETAILED DESCRIPTION

In the following description, numerous specific details are set forth in order to provide a thorough understanding of the present disclosure. It will be apparent, however, to one of ordinary skill in the art that the conductive eye mask skin treatment device and methods disclosed herein may be practiced without these specific details. In other instances, specific numeric references such as “first electrode,” may be made. However, the specific numeric reference should not be interpreted as a literal sequential order but rather interpreted that the “first electrode” is different than a “second electrode.” Thus, the specific details set forth are merely exemplary. The specific details may be varied from and still be contemplated to be within the spirit and scope of the present disclosure. The term “coupled” is defined as meaning connected either directly to the component or indirectly to the component through another component. Further, as used herein, the terms “about,” “approximately,” or “substantially” for any numerical values or ranges indicate a suitable dimensional tolerance that allows the part or collection of components to function for its intended purpose as described herein.

Electrostimulation is a well-known technology for non-invasive anti-aging treatments that enables users to age gracefully. Microcurrent treatments are known to increase the amount of adenosine triphosphate (ATP) within the cells of a muscle. Increases in cellular ATP levels have been shown to help muscles retain a re-educated form for longer periods of time, and as a consequence such techniques are of use in muscle toning treatments. In a salon environment, microcurrent electrostimulation devices typically include a pair of probes that can be used by a technician during a treatment to manipulate the skin and muscle such that the muscles are forced into a desired form for re-education. In a home environment, however, subjects desiring microcurrent treatments generally find themselves without the assistance of another person. Embodiments disclosed herein relate to an apparatus and methods for a conductive eye mask that may be coupled with a handheld electrostimulation skin treatment device for skin toning and firming that are intended for personally performing muscle toning treatments in absence of the assistance of another person.

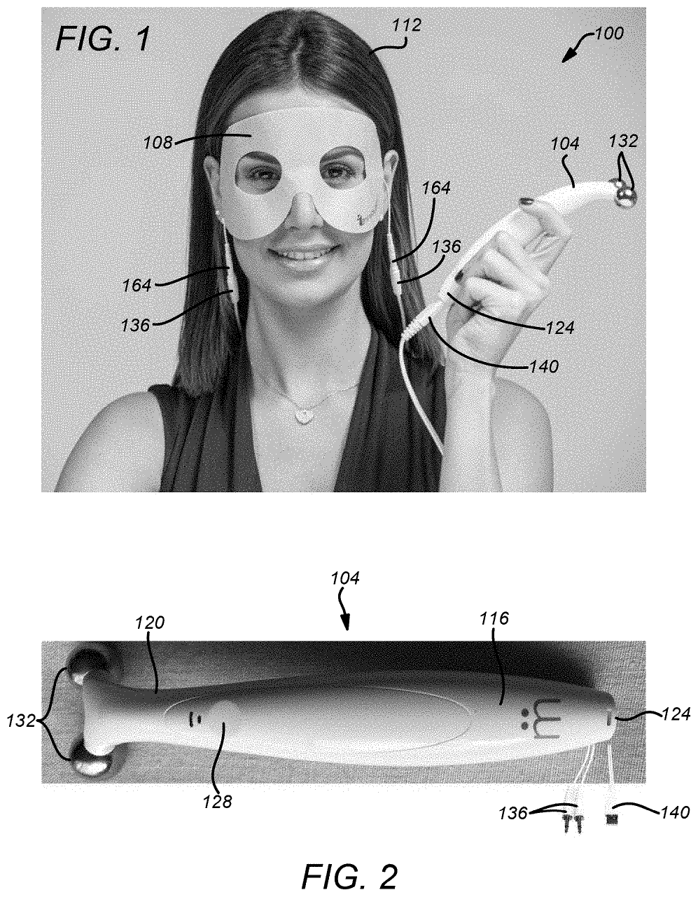

FIG. 1 illustrates an exemplary-use environment 100 wherein a handheld electrostimulation skin treatment device 104 is coupled with a conductive eye mask 108 that is applied to the face of a practitioner 112 for the purpose of treating the skin and muscles surrounding the eyes, in accordance with the present disclosure. As shown in FIG. 2, the treatment device 104 is a generally elongate member comprising a handle section 116 disposed between a treatment end 120 and an accessories end 124. The handle section 116 generally is adapted to be grasped in the hand of a practitioner using the device 104, as shown in FIG. 1. Further, the handle section 116 includes a power switch 128 that enables the practitioner to turn the treatment device 104 on and off, as desired.

The treatment end 108 includes at least two electrodes 132 disposed on opposite sides of the treatment end 120. The electrodes 132 are configured to supply electric current to the skin and muscles of the user, particularly but not exclusively to facial skin and muscles. Further, the electrodes 132 generally are configured to be pressed against the skin so as to manipulate the skin and muscles such that the muscles are forced into a desired form for re-education. As shown in FIGS. 1-2, the electrodes 132 comprise metallic spheres suitable for manipulating the skin and muscles of the user desiring a skin treatment. In some embodiments, a rechargeable battery and circuitry may be enclosed within the handle section 116 and configured to supply microcurrent electricity to the electrodes 132 when the power switch 128 is turned on.

As shown in FIG. 2, the accessories end 124 is adapted to receive any of various accessory connectors 136 as well as a Universal System Bus (USB) connector 140 that may be coupled with an external computing device, such as a smartphone or tablet PC. It is contemplated that, in some embodiments, the USB connector 140 may be configured to enable operating the treatment device 104 by way of a software application running on the external computing device. Meanwhile, as demonstrated in FIG. 1, the accessory connectors 136 facilitate coupling the treatment device 104 with an external accessory device, such as the conductive eye mask 108, that is configured for skin toning and firming as described herein.

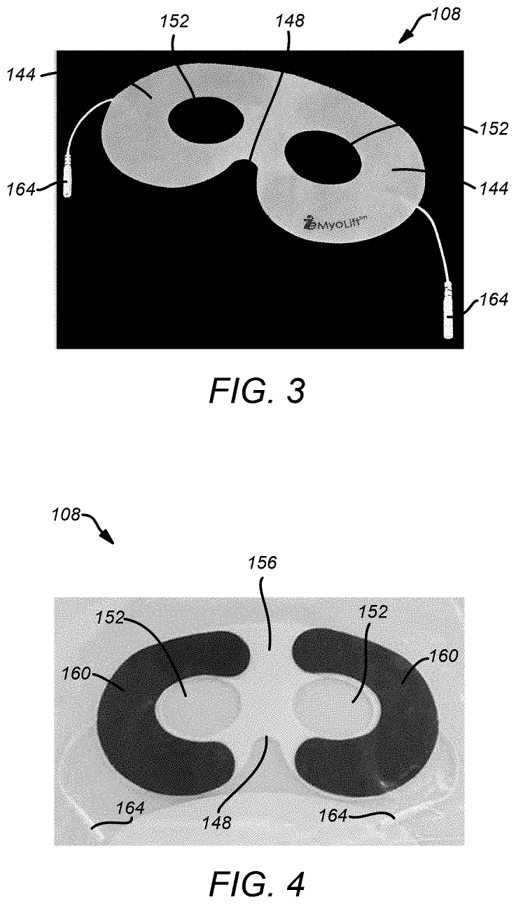

FIG. 3 illustrates a perspective view of an exemplary embodiment of a conductive eye mask 108 according to the present disclosure. The eye mask 108 is configured to be electrically coupled with the treatment device 104 for the purpose of treating the skin surrounding the eyes as shown in FIG. 1. The eye mask 108 includes round strips 144 that encircle the eyes and are joined at a nose bridge 148. The round strips 144 may be applied around the eyes of the practitioner 112 with the nose bridge 148 positioned above the practitioner's nose. An eye opening 152 is disposed within each round strip 144 to allow the practitioner 112 receiving treatment to see.

As shown in FIG. 4, an underside 156 of the eye mask 108 includes an electrode pad 160 that at least partially surrounds each of the eye openings 152. In the embodiment illustrated in FIG. 4, the electrode pads 160 are separated by the portion of the eye mask 108 between the eye openings 152. The degree of separation between the electrode pads 160 may be varied without limitation. It is contemplated that the electrode pads 160 may include a suitable adhesive for attaching to the skin of the practitioner 112. As will be appreciated, the electrode pads 160 are configured to pass an electric microcurrent from the device 104 to skin surrounding the practitioner's 112 eyes as shown in FIG. 1. To this end, each of the electrode pads 160 includes an electric connector 164 that is configured to be plugged into one of the accessory connectors 136 of the treatment device 104 shown in FIG. 1. The accessory connectors 136 and the electric connectors 164 cooperate to pass electric microcurrent from the device 104 to the electrode pads 160, enabling the practitioner 112 to personally perform muscle toning treatments to the region surrounding the practitioner's eyes in absence of the assistance of another person.

While the conductive eye mask skin treatment device and methods have been described in terms of particular variations and illustrative figures, those of ordinary skill in the art will recognize that the conductive eye mask skin treatment device is not limited to the variations or figures described. In addition, where methods and steps described above indicate certain events occurring in certain order, those of ordinary skill in the art will recognize that the ordering of certain steps may be modified and that such modifications are in accordance with the variations of the conductive eye mask skin treatment device. Additionally, certain of the steps may be performed concurrently in a parallel process when possible, as well as performed sequentially as described above. To the extent there are variations of the conductive eye mask skin treatment device, which are within the spirit of the disclosure or equivalent to the conductive eye mask skin treatment device found in the claims, it is the intent that this patent will cover those variations as well. Therefore, the present disclosure is to be understood as not limited by the specific embodiments described herein, but only by scope of the appended claims.

What is claimed is:

1. A conductive eye mask for treating the skin and muscles surrounding the eyes, comprising:
an eye mask for being applied around the eyes;
one or more electrode pads coupled with the eye mask; and
an electric connector coupled with each of the one or more electrode pads.

2. The conductive eye mask of claim 1, wherein the eye mask includes round strips configured to encircle the eyes and joined at a nose bridge.

3. The conductive eye mask of claim 2, wherein an eye opening is disposed within each round strip to allow a practitioner receiving treatment to see.

4. The conductive eye mask of claim 2, wherein the one or more electrode pads are attached to an underside of the eye mask such that each of the one or more electrode pads at least partially surrounds an eye opening.

5. The conductive eye mask of claim 1, wherein the one or more electrode pads includes an adhesive for attaching to skin of a practitioner receiving treatment.

6. The conductive eye mask of claim 1, wherein the electric connector is configured to be plugged into an accessory connector comprising an electrostimulation skin treatment device for sending a microcurrent to the one or more electrode pads.

7. A method for a conductive eye mask for treating the skin and muscles surrounding the eyes, comprising:
forming an eye mask for being applied around the eyes;
attaching one or more electrode pads with the eye mask; and
coupling an electric connector with each of the one or more electrode pads.

8. The method of claim 7, wherein forming includes configuring round strips to encircles the eye and joining the round strip at a nose bridge.

9. The method of claim 7, wherein attaching includes affixing the one or more electrode pads to an underside of the eye mask such that each of the one or more electrode pads at least partially surrounds an eye opening.

10. The method of claim 7, wherein coupling includes configuring the electric connector to be plugged into an accessory connector comprising an electrostimulation skin treatment device for sending a microcurrent to the one or more electrode pads.

11. A method for treating skin and muscles surrounding the eyes of a practitioner, comprising:
applying an eye mask around the eyes;
attaching one or more electrode pads of the eye mask to the skin;
coupling the one or more electrode pads with an electrostimulation skin treatment device; and
applying a microcurrent to the one or more electrode pads.

12. The method of claim 11, wherein applying includes encircling the eyes with round strips comprising the eye mask such that the practitioner can see through eye openings disposed in the eye mask.

13. The method of claim 11, wherein attaching includes using a suitable adhesive to hold the one or more electrode pads in contact with the skin.

14. The method of claim 11, wherein coupling includes plugging the one or more electrode pads into the electrostimulation skin treatment device.

15. The method of claim 14, wherein plugging includes coupling an electric connector comprising the eye mask into an accessory connector comprising an electrostimulation skin treatment device.

16. The method of claim 15, wherein an electric connector is coupled with each of the one or more electrode pads.
